(19) United States
(12) Patent Application Publication
(10) Pub. No.: US 2014/0288328 A1
(43) Pub. Date: Sep. 25, 2014

(54) SEPARATION TECHNIQUE, PHOTO-OXIDATION OF ORGANIC SUBSTRATES, AND PHOTO CATALYSTS

(71) Applicant: The University of Nottingham, Nottinghamshire (GB)

(72) Inventors: Martyn Poliakoff, Nottingham (GB); Mike W. George, Nottingham (GB); Richard A. Bourne, Nottingham (GB); Xue Han, Nottingham (GB); Jessica F. B. Hall, Nottingham (GB)

(21) Appl. No.: 14/358,578

(22) PCT Filed: Nov. 13, 2012
(86) PCT No.: PCT/GB2012/052818

§ 371 Date: May 15, 2014

(30) Foreign Application Priority Data
Nov. 15, 2011 (GB) 1119679.7

(51) Int. Cl. C07C 51/25; B01J 19/12

(52) U.S. Cl. CPC C07C 51/25; B01J 19/122
USPC 562/538; 204/157.15; 210/638

(57) ABSTRACT

A method for photo-oxidising an organic substrate to form an organic product is disclosed comprising: a) mixing oxygen, a supercritical fluid, a photocatalyst, a liquid fluorous solvent and an organic substrate to form a mixture; and b) irradiating the mixture to form an organic product. Also disclosed is a method for separating a photocatalyst from an organic product comprising the steps of: a) providing a mixture comprising a supercritical fluid; an organic product; a fluorous solvent; a photocatalyst; and optionally an organic substrate and optionally oxygen; wherein the organic product, fluorous solvent, photocatalyst and optional organic substrate and optional oxygen are dissolved in the supercritical fluid; and b) reducing the pressure of the mixture to a pressure below the critical pressure of the supercritical fluid in order to form a gaseous phase.

### FIELD OF THE INVENTION

The present invention relates to the photo-oxidation of organic substrates and, in particular, continuous photo-oxidation with singlet oxygen in a supercritical fluid, preferably supercritical carbon dioxide, by use of fluorous biphasic catalysis. The invention relates to methods of photo-oxidising organic substrates, separating photocatalysts from organic products, mixtures for use in photo-oxidation and methods of manufacturing photocatalysts, new photo-catalysts, and new uses thereof.

### BACKGROUND OF THE INVENTION

Photochemically generated singlet oxygen, ¹O₂, can provide a clean and sustainable route to photo-oxidised compounds. In the current regulatory climate, however, difficulties arise in selecting suitable solvents for the scale-up of such photo-oxidations due to the highly reactive nature of ¹O₂. Traditionally, these reactions have been performed in chlorinated solvents, e.g. CCl₄, chosen because of their non-flammability and the long lifetime of ¹O₂ in these solvents. Nowadays, such solvents are unacceptable for most commercial applications because of their toxicity and environment impact. Therefore new approaches are needed so that ¹O₂ can realise its full potential in the Green Chemistry tool-box.

Supercritical carbon dioxide, scCO₂, has been explored as an alternative solvent, enabling photo-oxidations to be conducted safely in a non-flammable and non-toxic solvent. ¹O₂ has a relatively long lifetime in scCO₂ and its reactions occur more rapidly than in traditional solvents because single phase supercritical conditions reduce mass transfer limitations. Nevertheless, there are still problems because the high pressures of scCO₂ dictate that any commercially viable scale-up generally must involve continuous processes. The problems arise because ¹O₂ often requires the use of a photocatalyst. These photocatalysts can be immobilised but they and their support need to be stable under both irradiative and high pressure conditions.

Photocatalysts immobilised on a solid polymer support in continuous scCO₂ reactors are known. However, the catalyst lifetime is often found to be limited <10 hours by degradation of the polymer support, predominantly by plasticisation, under irradiation and supercritical conditions; which eventually blocks the reactor. Much more preferable would be the feeding of homogeneous catalysts continuously into the reactor but these catalysts are difficult to separate from the reaction products, which almost invariably are thermally sensitive if not potentially explosive.

There is therefore a need for photocatalysts and methods for photo-oxidising, and in particular methods for photo-oxygenating, an organic substrate which are cheaper, more efficient, safer and more environmentally friendly than those known in the art.

The invention sets out to address these and other problems with the prior art.

### SUMMARY OF THE INVENTION

In a first aspect, the present invention provides a method for photo-oxidising an organic substrate to form an organic product. Typically, the method is a method for photo-oxygenating an organic substrate to form an organic product. Preferably, the method comprises the steps of: a) mixing oxygen, a supercritical fluid, a photocatalyst, a fluorous solvent and an organic substrate to form a mixture; and, preferably, b) irradiating the mixture to form an organic product. Typically, the supercritical fluid is supercritical carbon dioxide.

Such a method allows the homogenous photo-catalysis of organic substrates while addressing the problems with the prior art.

In an embodiment, following the irradiation step, the method further comprises the step of reducing the mixture pressure to below the critical pressure of the supercritical fluid to form a gaseous phase. Typically, in order to form the gaseous phase and liquid fluorous solvent. Preferably, the fluorous solvent is liquid at the critical point of the supercritical fluid. Preferably, the fluorous solvent is liquid at pressures at or below the critical pressure of the supercritical fluid. Preferably, the fluorous solvent is liquid at standard temperature and pressure (i.e. 20° C. and an absolute pressure of 101.325 kPa). Typically, the gaseous phase is gaseous carbon dioxide. Typically, the pressure is lowered below the critical pressure of carbon dioxide, preferably to atmospheric pressure (i.e. 101.325 kPa). Typically, the temperature will be kept constant, although it is possible for the temperature to be raised or, more preferably, lowered, typically below the critical temperature of the supercritical fluid, typically to below the critical temperature of carbon dioxide, typically to room temperature (i.e. 20° C.). Preferably, the phase separation of the supercritical fluid and fluorous solvent is controlled by varying the pressure. Controlling the phase separation of the supercritical fluid and fluorous solvent using pressure, rather than relying on temperature modulated extraction techniques, is preferable because it is safer for this type of compound.

In a further embodiment, following the step of reducing the mixture pressure to below the critical pressure of the supercritical fluid to form a gaseous phase, the method preferably comprises the step of separating the gaseous phase and fluorous solvent; typically, gaseous carbon dioxide and liquid fluorous solvent. This is typically followed by the step of separating the fluorous solvent from the organic product.

It has been found by the inventors that it is sufficiently easy to separate fluorous solvents from the gaseous phase, and in particular carbon dioxide, that the method can be used as part of a continuous homogenous catalysis reaction process in which the fluorous solvent and photo-catalyst are recycled. Pressure modulated separation of carbon dioxide and fluorous solvent is advantageous as it is safer than temperature modulated extraction methods.

In a still further embodiment, the organic product and liquid fluorous solvent are substantially immiscible and/or insoluble. Preferably, organic product and liquid fluorous solvent separate into distinct phases, preferably distinct liquid phases. Preferably, the fluorous solvent is denser than the organic product. Preferably, the organic product is more polar than the fluorous solvent. By having some or all of these properties it is relatively simple to extract the fluorous solvent from the organic product.

In embodiments, the organic product may be diluted during the separation step using a non-fluorous solvent in order to lower the viscosity of the organic product. Suitable non-fluorous solvents include methanol. In embodiments, the fluorous solvent and organic product may be agitated in order to expedite separation, for instance by using an ultrasonic bath.

In embodiments, the photocatalyst is soluble in the fluorous solvent. Preferably, the photocatalyst is preferentially soluble in the fluorous solvent over the organic product.

In a further embodiment, when the fluorous solvent is separated from the organic product, the fluorous solvent comprises photocatalyst from the mixture, preferably a majority of the photocatalyst from the mixture, preferably at least about 70% of the photocatalyst from the mixture, preferably at least about 80% of the photocatalyst from the mixture, more preferably substantially all of the photocatalyst.

By having some or all of these properties it is relatively simple to extract separate the photocatalyst from the organic product. Easy extraction of the photocatalyst is beneficial as because the photocatalyst often represents the most valuable component of the mixture.

Typically, the photocatalyst, organic substrate, organic product, and fluorous solvent are soluble in the supercritical fluid, preferably supercritical carbon dioxide.

In preferred embodiments, the fluorous solvent and the photocatalyst are recycled. This has the advantage of greatly reducing the amount of photocatalyst required to provide a given amount of organic product. Typically, any fluorous solvent lost when removing the gaseous carbon dioxide may be replaced. Preferably, substantially all of the fluorous solvent is recovered from the gaseous carbon dioxide, e.g. by distillation.

In preferred embodiments, the mixture is single phase, preferably a clear single phase. Preferably, the mixing step and/or irradiating steps are conducted at a pressure sufficient for the mixture to form a single phase, preferably a clear single phase. Typically, the pressure is at least about 7.38 MPa; preferably, at least about 14 MPa; more preferably, at least about 16 MPa. The skilled person would be able to select an appropriate preferred pressure by observing when the mixture transitions from a cloudy to a clear single phase and selecting a pressure above that of the transition. The pressure will be above the critical pressure of the supercritical fluid. The mixing and/or irradiating steps are conducted at a temperature above the critical temperature of the supercritical fluid. Typically, the temperature of the mixture is at least about 31.1° C.; preferably, at least about 40° C.; preferably, from about 31.1° C. to about 70° C.; preferably, from about 40° C. to about 60° C., preferably about 55° C.

In preferred embodiments, the photocatalyst is fluorophilic, preferably a fluorinated photocatalyst. Fluorinated photocatalysts are preferred as they show good solubility in supercritical fluids, in particular carbon dioxide, and fluorous solvents. Known photocatalysts may be fluorinated by reacting them with a fluorinating agent, for instance with a with a fluoroalkanethiol, preferably a perfluoroalkanethiol, such as 3,3,4,4,5,5,6,6,7,7,8,8,9,9,10,10,10-heptadecafluorodecane-1-thiol. Other preferred fluorinating agents include perfluoroalkaneamines and amine and thiol benzyl substituted perfluoroalkanes such as 3-perfluorooctylpropylamine, 4-methyl-3,5-bis(perfluorohexyl)benzylthiol, 4-methyl-3,5-bis(perfluorohexyl)benzylamine.

In embodiments, the photocatalyst is a fluorinated derivative of a photocatalyst; examples preferably include organic non-polar sensitisers such as porphyrin, chlorin, bacteriochlorin, phthalocyanine, or fullerene (C₆₀). Other embodiments use polar sensitisers with appropriate fluorinated ions, suitable examples include methylene blue, rose Bengal, Ru²⁺L₃ complexes, where L is any combination of bipyridine, bipyrazine, and 2,2-bipyrimidine and derivatives thereof.

In embodiments, the photocatalyst is selected from the group consisting of non-polar organic sensitisers, typically porphyrins.

In a preferred embodiment, the a photocatalyst is

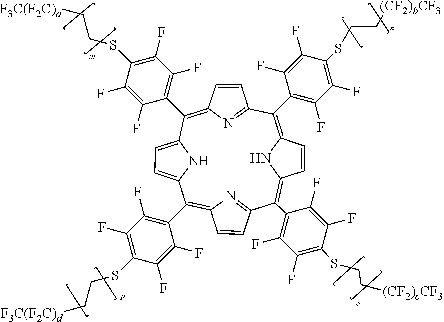

wherein a, b, c and d=≧1, preferably, from 1 to 16, more preferably, from 2 to 10, more preferably, 7; and m, n, o, p≧1, preferably, 1.

Preferably, a=b=c=d. Preferably, m=n=o=p.

A particularly preferred photocatalyst is

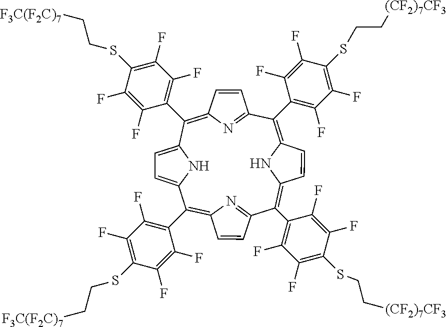

In further embodiments, the organic substrate is selected from the group consisting of unsaturated hydrocarbons, preferably dienes (particularly alpha-terpinene), furans, highly substituted olefins (particularly tetra or tri substituted e.g. citronellol), allylic alcohols, carbonyl compounds.

In embodiments, the fluorous solvent is selected from the group consisting of fluorocarbons and fluoroethers and combinations thereof, preferably perfluorocarbons, hydrofluorocarbons, perfluoroaromatics, perfluorethers, and hydrofluoroethers.

Suitable perfluorocarbons include perfluorohexane, perfluoromethylcyclohexane, or perfluorodecalin.

Suitable hydrofluoroethers (HFEs) include HFE-7100 and HFE-7500 (both sold by 3M).

In a second aspect, the invention provides an organic product manufactured using any of the methods of the first aspect.

In a third aspect, the invention provides the use of the method of the first aspect in the manufacture of trioxane, and derivatives thereof, primarily hydroxyl substituted hydroperoxides preferentially possessing a hydroxyl group located 3-4 bonds from the peroxide, possibly 2-5 bonds.

In a fourth aspect, the invention provides a mixture comprising an organic substrate and/or an organic product; optionally oxygen; a supercritical fluid; a photocatalyst and a fluorous solvent. Preferably, the supercritical fluid is supercritical carbon dioxide. Typically, when there is no organic substrate in the mixture, the mixture will not comprise oxygen. Typically, the mixture is single phase; preferably, the mixture is a clear single phase. Typically, the mixture will transition from a multi-phase to a single-phase mixture by increasing the pressure of the mixture. Typically, the pressure is at least about 7.39 MPa; preferably, at least about 14 MPa; more preferably, at least about 16 MPa. The skilled person would be able to select an appropriate preferred pressure by observing when the mixture transitions from a cloudy to a clear single phase. Typically, the temperature of the mixture is at least about 31.1° C.; preferably, at least about 40° C.; preferably, from about 31.1° C. to about 70° C.; preferably, from about 40° C. to about 60° C., preferably about 55° C.

The invention also provides the use of the mixture of the fourth aspect in the photo-oxidation, preferably photo-oxygenation, of an organic substrate.

The invention also provides the use of the mixture of the fourth aspect in a homogenous catalysis photo-oxidation reaction; preferably a homogenous catalysis photo-oxygenation reaction; preferably, wherein the photocatalyst is recycled.

The photocatalysts, fluorous solvents, organic products and organic substrates described in embodiments of the first aspect are also applicable to the second, third and fourth aspects of the invention.

In a fifth aspect, the present invention provides a photocatalyst

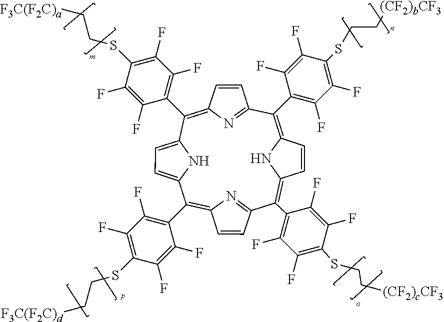

wherein a, b, c and d≧1; preferably; from 1 to 16; preferably from 2 to 10; preferably, 7; and m, n, o and p≧1; preferably, 1. When m=n=o=p=1 and a=b=c=d, a≠9.

Preferably, a=b=c=d. Preferably, m=n=o=p.

A particularly preferred photocatalyst is

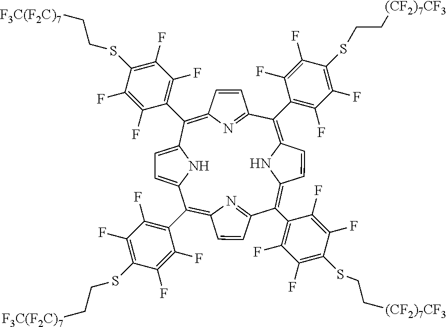

The invention also provides the use of the photocatalyst of the fifth aspect in homogenous catalysis.

The invention further provides the use of the photocatalyst of the fifth aspect of the invention in a photo-oxidation reaction, preferably, a photo-oxygenation reaction; preferably continuous homogenous catalysis photo-oxidation reaction, more preferably in a continuous homogenous catalysis photo-oxygenation reaction.

The invention also provides the use of the photocatalyst of the fifth aspect of the invention in any of the methods of the first aspect of the invention.

In a sixth aspect, the invention provides a method for manufacturing a photocatalyst according to the fifth aspect comprising the step of reacting 5,10,15,20-tetrakis(pentafluorophenyl)porphyrin (TPFPP) with a fluoroalkanethiol, preferably, a perfluoroalkanethiol, preferably, 3,3,4,4,5,5,6,6,7,7,8,8,9,9,10,10,10-heptadecafluorodecane-1-thiol. In certain embodiments, a mixture of different fluoroalkanethiols may be used. Other preferred fluorinating agents include perfluoroalkaneamines and amine and thiol benzyl substituted perfluoroalkanes such as 3-perfluorooctylpropylamine, 4-methyl-3,5-bis(perfluorohexyl)benzylthiol, 4-methyl-3,5-bis(perfluorohexyl)benzylamine.

In a seventh aspect, the invention provides a method for manufacturing a photocatalyst for use in the homogenous catalysis of an organic substrate comprising the steps of a) selecting a fluorous solvent; b) selecting a supercritical fluid; c) selecting a photocatalyst; and d) fluorinating the photocatalyst to such an extent that the fluorinated photocatalyst is soluble in both the fluorous solvent and the supercritical fluid.

Preferably, the supercritical fluid is supercritical carbon dioxide. The fluorous solvents, photocatalysts and organic substrates may be selected from any of those listed in relation to the previous aspects and embodiments of the invention. Preferably, the fluorinated photocatalyst is preferentially soluble in the fluorous solvent over the photo-oxidised organic substrate.

The invention also provides a photocatalyst manufactured according to the seventh aspect of the invention, and the use of said photocatalyst in the photo-oxidation of an organic substrate.

In an eighth aspect the invention provides a method for separating a photocatalyst from an organic product comprising the steps of: a) providing a mixture comprising a supercritical fluid; an organic product; a fluorous solvent; a photocatalyst; and optionally an organic substrate and optionally oxygen; wherein the organic product, fluorous solvent, photocatalyst and optional organic substrate and optional oxygen are dissolved in the supercritical fluid; and b) reducing the pressure of the mixture to a pressure below the critical pressure of the supercritical fluid in order to form a gaseous phase.

All aspects of the method of first aspect of the invention may be combined mutatis mutandis with the eighth aspect of the invention.

In all of the aspects and embodiments of the invention, photo-oxidation includes photo-oxygenation.

### BRIEF DESCRIPTION OF THE FIGURES

The above-mentioned and other features, embodiments and aspects of this invention, and the manner of obtaining them, will become more apparent and the invention itself will be better understood by reference to the following description of embodiments of the invention taken in conjunction with the accompanying drawings, wherein:

FIG. 1 is a series of photographs showing the phase behaviour of a α-terpinene (6.4 mmol)+HFE-7500 (4.0 mmol)+CO₂ (0.24 mol)+N₂ (12.5 mmol) system at 55° C. under different pressure.

FIG. 2 is a series of photographs showing the phase behaviour a citronellol (5.5 mmol)+HFE-7500 (3.9 mmol)+CO₂ (0.24 mol)+N₂ (12.5 mmol) system at 55° C. under different pressure.

FIG. 3 is a graph showing conversion/pressure for a α-terpinene (6.4 mmol)+HFE-7500 (4.0 mmol)+CO₂ (0.24 mol)+N₂ (12.5 mmol) system at 55° C. under different pressure.

FIG. 4 is a graph showing conversion/pressure for a citronellol (5.5 mmol)+HFE-7500 (3.9 mmol)+CO₂ (0.24 mol)+N₂ (12.5 mmol) system at 55° C.

FIG. 5 is a schematic of the continuous recycle experiment equipment.

### DETAILED DESCRIPTION OF THE INVENTION

The present invention provides a method for the photo-oxidation of an organic substrate to an organic product comprising the steps of: a) mixing oxygen, a supercritical fluid, a photocatalyst, a fluorous solvent and an organic substrate to form a mixture; and, preferably, b) irradiating the mixture to form an organic product. Typically, the supercritical fluid is supercritical carbon dioxide.

Supercritical fluid is the fluid state of any substance at a temperature and pressure above its critical point, where distinct liquid and gas phases do not exist. Typically, supercritical fluids can effuse through solids like a gas, and dissolve materials like a liquid.

Suitable supercritical fluids for use in the invention include supercritical methane, ethane and carbon dioxide.

Supercritical carbon dioxide is a fluid state of carbon dioxide where it is held at or above its critical temperature and critical pressure. Specifically, carbon dioxide behaves as a supercritical fluid above its critical temperature of 31.1° C. and critical pressure of 7.38 MPa.

For the purpose of the invention, photo-oxidation is understood to be a light-induced oxidation reaction. Photo-oxidation includes photo-oxygenation.

For the purpose of the invention, photo-oxygenation is understood to be a light-induced oxidation reaction in which molecular oxygen is incorporated into organic substrate. Typically, the photo-oxygenation reactions of the invention use singlet oxygen (¹O₂).

Photocatalyst is understood to mean a catalyst which accelerates a photo-oxidation, preferably photo-oxygenation, reaction. Preferably, the photocatalyst is soluble in the fluorous solvent. Preferably, the photocatalyst is preferentially soluble in the liquid fluorous solvent over the organic product. Preferably, the photocatalyst is soluble in supercritical carbon dioxide. Relative solubility should be measured at standard temperature and pressure.

Preferably, the photocatalyst is fluorophilic. Preferably the photocatalyst is fluorinated. Fluorinated photocatalysts are preferred as they show good solubility in supercritical carbon dioxide and fluorous solvents. Known photocatalysts may be fluorinated by reacting them with a fluorinating agent, for instance a with a fluoroalkanethiol, preferably a perfluoroalkanethiol, such as 3,3,4,4,5,5,6,6,7,7,8,8,9,9,10,10,10-heptadecafluorodecane-1-thiol. Other preferred fluorinating agents include perfluoroalkaneamines and amine and thiol benzyl substituted perfluoroalkanes such as 3-perfluorooctylpropylamine, 4-methyl-3,5-bis(perfluorohexyl)benzylthiol, 4-methyl-3,5-bis(perfluorohexyl)benzylamine.

In embodiments, the photocatalyst is a fluorinated derivative of a photocatalyst examples preferably include organic non-polar sensitisers such as porphyrin, chlorin, or bacteriochlorin, phthalocyanine, fullerene (C₆₀). Other embodiments use polar sensitisers with appropriate fluorinated ions, suitable examples include methylene blue, rose Bengal, Ru²⁺L₃ complexes, where L is any combination of bipyridine, bipyrazine, and 2,2-bipyrimidine and derivatives thereof.

In a preferred embodiment, the a photocatalyst is

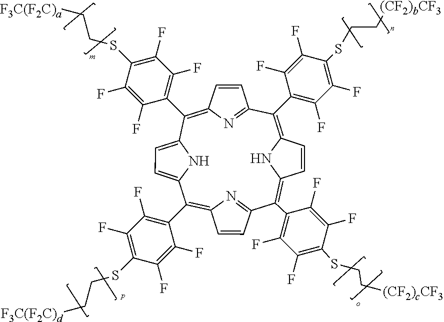

wherein a, b, c and d=≧1, preferably, from 1 to 16, more preferably, from 2 to 10, more preferably, 7; and m, n, o, p≧1, preferably, 1.

Preferably, a=b=c=d. Preferably, m=n=o=p.

Said preferred photocatalysts can be synthesised by reacting 5,10,15,20-tetrakis(pentafluorophenyl)porphyrin (TPFPP) with appropriate fluoroalkanethiols.

A particularly preferred photocatalyst is

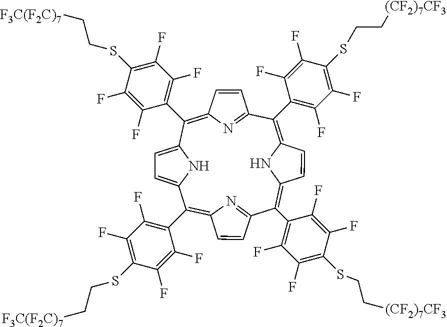

Said particularly preferred photocatalyst can be synthesised by reacting 5,10,15,20-tetrakis(pentafluorophenyl)porphyrin (TPFPP) (available from Sigma Aldrich) with 3,3,4,4,5,5,6,6,7,7,8,8,9,9,10,10,10-heptadecafluorodecane-1-thiol (Sigma Aldrich). Typically, the reaction requires the presence of a secondary amine, preferably diethylamine (Sigma Aldrich). Preferred solvents for use in the reaction include ethyl acetate (Sigma Aldrich) and dimethylformamide (Sigma Aldrich), and combinations thereof. Other appropriated solvents will be apparent to the person skilled in the art. When used in combination, preferably the ethyl acetate and dimethylformamide are used in a volume to volume ratio of from about 5:1 to about 1:5; preferably from about 2:1 to about 1:2; preferably about 2:1.

The organic substrate may be any capable of being photo-oxidised or photo-oxygenated.

Preferred organic substrates include tetrahydropyran and derivatives thereof.

Particularly preferred organic substrates are precursors to artemisinin and derivatives thereof, including precursors to artesunate, dihydroartemisinin, artelinic acid, artenimol, arterolane and artemotil.

Organic product may be any photo-oxidised or photo-oxygenated organic substrate.

Preferred organic products include trioxane, and derivatives thereof, and in particular anti-malarial trioxanes.

Particularly preferred organic products are anti-malarial trioxanes selected from the group consisting of artemisinin, artesunate, dihydroartemisinin, artelinic acid, artenimol, arterolane and artemotil. Such organic products may be used alone, or in combination, in the treatment of malaria. They may also be used in combination with other anti-malarial active pharmaceutical ingredients (APIs) in the treatment of malaria. Other anti-malarial APIs suitable for use in combination with the above organic products include quinine, mefloquine, amodiaquine, lumefantrine, piperaquine.

Suitable fluorous solvents include fluorocarbons and fluoroethers, preferably perfluorocarbons, perfluoroaromatics, hydrofluorocarbons, perfluorethers, and hydrofluoroethers. Preferably, fluorous solvents do not include chlorofluorocarbons.

Suitable perfluorocarbons include perfluorohexane, perfluoromethylcyclohexane, or perfluorodecalin.

Suitable hydrofluoroethers (HFEs) include nonafluorobutyl methyl ether, sold under the trade name HFE-7100 by 3M, and HFE-7500, also available from the 3M. These solvents retain many of the fluorous characteristics of perfluorocarbons, but possess higher overall solvating power due to increased polarity relative to perfluorocarbons. They also have less ozone depletion potential due to their short atmospheric lifetime.

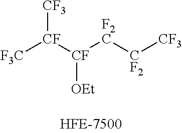

Irradiating the mixture typically comprises exposing the mixture to light, which for the purposes of the invention typically includes infra-red, ultraviolet and visible light. Preferably, the light has a wavelength of from about 170 nm to about 300 μm; preferably, from about 200 nm to about 2,500 nm; preferably from about 380 nm to about 760 nm. An appropriate light source will typically be selected depending on the wavelength and intensity of light required. The wavelength of light will typically be determined by choice of photocatalyst; the wavelength being that required for the photocatalyst to catalyse the photo-oxidation of the organic substrate. White light sources, as well as narrow band or single wavelength sources, may be used in the invention for irradiating the mixture. A filter may also be used to select the wavelength required. The light source may be any appropriate light source, although light emitting diodes (LEDs) are preferred because of their low energy consumption.

### EXAMPLES

### 1. Synthesis of the Photocatalyst

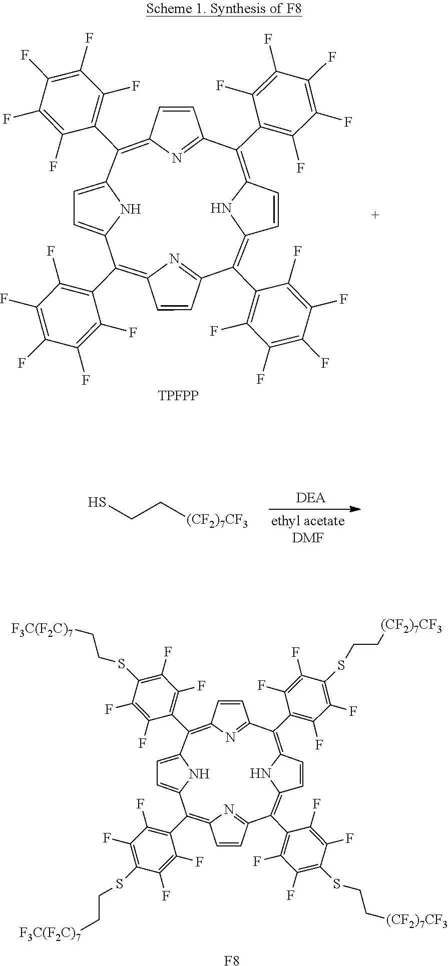

Highly fluorinated photocatalyst, referred to herein as F8, was synthesised following Scheme 1.

Specifically, 3,3,4,4,5,5,6,6,7,7,8,8,9,9,10,10,10-heptadecafluorodecane-1-thiol (325 mg, 0.68 mmol) (Sigma Aldrich) was dissolved in 15 mL ethyl acetate (Sigma Aldrich)/dimethylformamide (DMF) (Sigma Aldrich) (2:1 v/v) with diethylamine (DEA) (0.1 mL, 0.97 mmol) (Sigma Aldrich) under nitrogen. 5,10,15,20-tetrakis(pentafluorophenyl)porphyrin (TPFPP) (57.5 mg, 0.06 mmol) dissolved in 5 mL DMF was added to this solution. The resulting solution was stirred under nitrogen at room temperature for 8 hours. The solution underwent centrifugation (8000×g) for 15 minutes. The solid was isolated by filtration, and re-dissolved in acetone and purified by silica gel chromatography using hexane/acetone (9:1 v/v). The yield of F8 was 112 mg (0.04 mmol, 67%). The obtained F8 was characterised by ¹H and ¹⁹F-NMR. ¹H-NMR (CDCl₃ 5% TFA) δ: −2.84 (broad s, 2H, pyrrole NH), 2.64 to 2.81 (m, 8H, 2′H), 3.50 (t, 8H, 1′H), 8.94 (s, 8H, pyrrole βH). ¹⁹F-NMR: (CDCl₃ 5% TFA) δ: −136.12 to −135.99 (m, 8F, Ar-o-F), −133.69 to −133.56 (m, 8F, Ar-m-F), −126.18 to −126.08 (m, 8F, 3′F), −123.14 (m, 8F, 4′F), −122.71 (m, 8F, 5′F), −121.68 to −121.61 (m, 24F, 6′-8′F), −113.86 to −113.75 (m, 8F, 9′F), −80.83 to 80.76 (m, 12F, 10′F).

This method can be modified to fluorinate alternative photocatalysts. Furthermore, the fluoroalkanethiol of the method can be varied in order to obtain photocatalysts with alternative fluorinated chains.

### 2. Phase Behaviour

Phase behaviour of the α-terpinene (6.4 mmol)+HFE-7500 (4.0 mmol)+CO₂ (0.24 mol)+N₂ (12.5 mmol) system was studied using a variable volume view cell. Molar ratios of all the components were chosen to mimic the continuous flow experiment described below. N₂ has very similar properties to O₂ in terms of phase behaviour under this condition, it is therefore an acceptable substitute for O₂ and avoids any potential safety hazards associated with O₂.

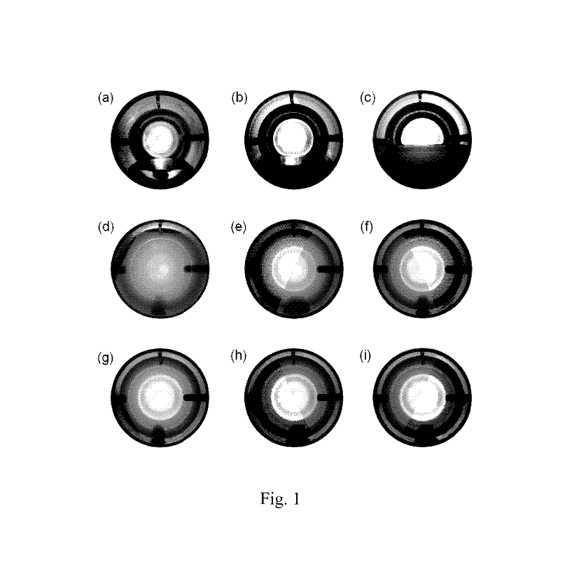

FIG. 1 contains photographs which show the phase behaviour of the above mixture at 55° C. under different pressure. FIG. 1: (a) 10 Mpa, multi-phase, (b) 11 MPa, multi-phase (c) 12 MPa, multi-phase, (d) 13 MPa, cloudy single phase, (e) 14 MPa, cloudy single phase, (f) 15 MPa, cloudy single phase, (g) 16 MPa, clear single phase, (h) 17 MPa, clear single phase, (i) 18 MPa, clear single phase.

The phase behaviour of the citronellol (5.5 mmol)+HFE-7500 (3.9 mmol)+CO₂ (0.24 mol)+N₂ (12.5 mmol) system at 55° C. under different pressure was also investigated.

FIG. 2 shows the results. FIG. 2: (a) 10 MPa, multi-phase, (b) 11 MPa, multi-phase (c) 12 MPa, multi-phase, (d) 13 MPa, multi-phase, (e) 14 MPa, cloudy single phase, (f) 15 MPa, cloudy single phase, (g) 16 MPa, clear single phase, (h) 17 MPa, clear single phase, (i) 18 MPa, clear single phase.

HFE-7500 is available from 3M.

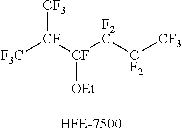

### 3. Initial Flow Experiments

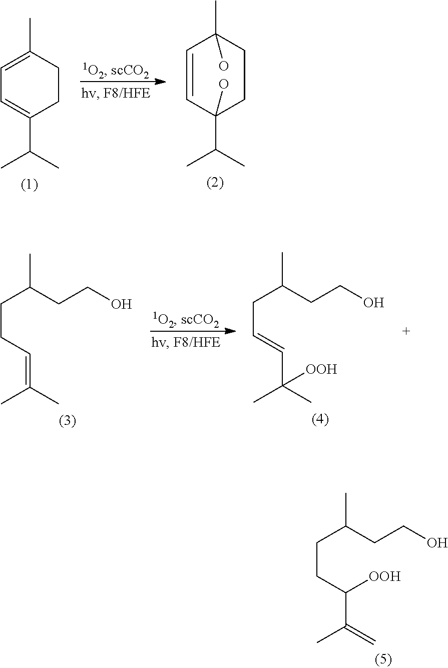

Initial flow experiments investigated the effect of pressure on the reaction of α-terpinene (1) and the biphasic separation of the organic product (2). It was found that the reaction was single-phase at >14 MPa with >99% conversion. Although ca. 50% of the HFE-7500 was vented with the gaseous CO₂, none of (2) was lost. The liquid HFE-7500 and (2) showed clear phase separation but some F8 remained in the (2). However, much of this F8 could be recovered from (2) by separating the layers, leaving them to stand, and then adding enough HFE-7500 to make up the loss during venting.

The continuous photo-oxidation of citronellol (3) was also investigated. A single phase system was observed at >16 MPa with >99% conversion. As the product mixture was rather viscous but, (3), (4) and (5) are significantly more polar than either (1) or (2), less F8 was left in the organic phase than with (2).

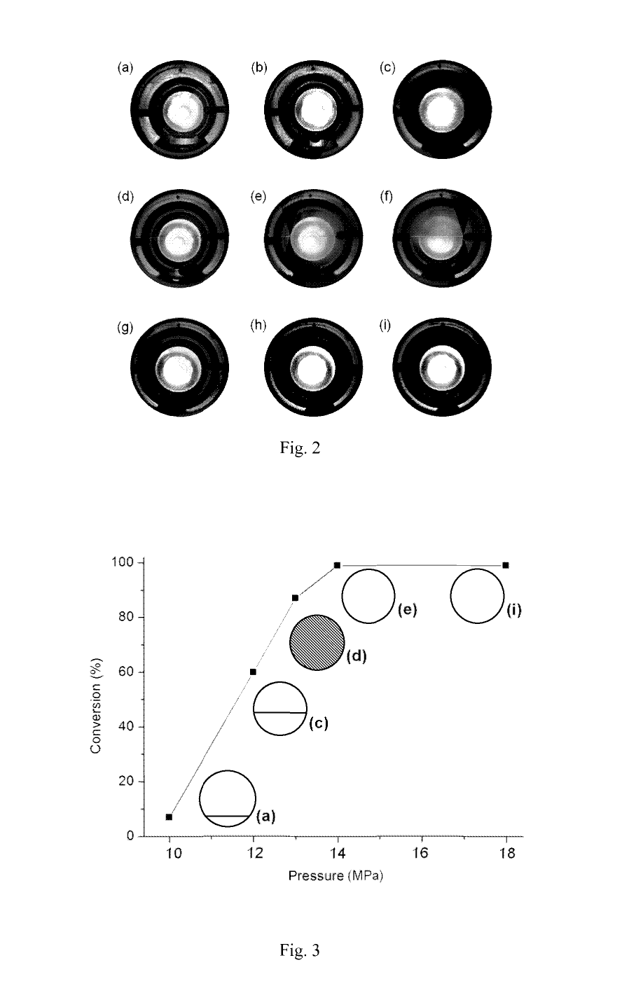

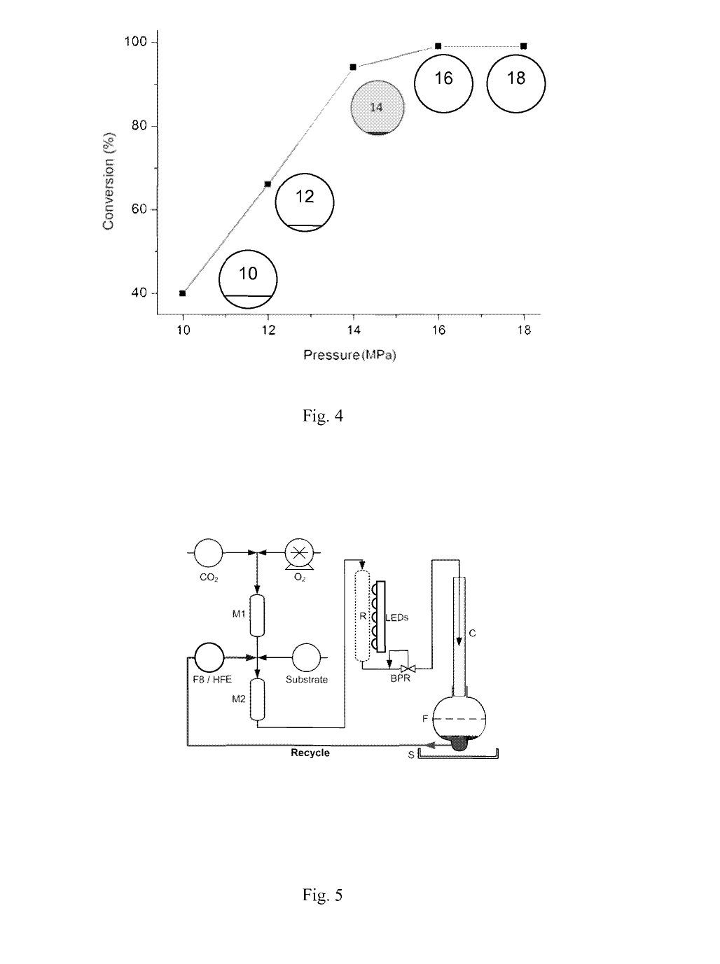

FIG. 3 and FIG. 4 show the results of the initial flow experiments for α-terpinene and citronellol respectively. FIG. 3 and FIG. 4 also include schematic representations of the phase behaviour results from FIG. 1 and FIG. 2 respectively.

### 4. Continuous Recycle Experiment

A fully continuous recycle of the F8/HFE-7500 was then carried out. F8/HFE-7500 and citronellol (3) were pumped separately and the fluorous phase (i.e. F8/HFE-7500) in the product flask acted as the reservoir for the F8/HFE-7500 pump. Methanol was added periodically to the product collection flask to lower the viscosity of the organic product and an aliquot of HFE-7500 was added each hour to replace the HFE-7500 lost during depressurisation. The flow rates were increased to force the conversion of (3) to around 50% by decreasing the residence time while still maintaining a single phase in the reactor. Under these stressed conditions, the effect of recycling F8/HFE-7500 on the conversion during the reaction could be investigated.

FIG. 5 shows a schematic of the photochemical reactor with continuous fluorous phase (i.e. F8/HFE-7500) recycling. CO₂ is delivered by a Jasco PU-1580-CO₂ pump, O₂ is added using a Rheodyne dosage unit. The citronellol and F8/HFE-7500 are simultaneously pumped using two Jasco PU-980 HPLC pumps. M1 and M2: mixers; R: sapphire tube reactor; LEDs: light source; BPR: back-pressure regulator (Jasco BP-1580-81); C: glass condenser; F: glassware flask; S: ultrasonic bath to accelerate phase separation of the product and F8/HFE-7500.

12 mL of F8 in HFE-7500 were recycled for ca. 20 h, at a flow rate of 0.1 mLmin⁻¹. Citronellol (3) was pumped at 0.2 mLmin⁻¹; pressure 18 MPa.

Table 1 compares the photo-oxidation of citronellol with the fluorous biphasic system, HFE-7500/F8, with the continuous reaction with dimethylcarbonate (DMC)/TPFPP described in Angew. Chem. Int. Ed. Engl., 2009, 48, 5322. Bearing in mind that the F8/HFE-7500 experiment was deliberately run to give only 50% conversion, one can see that fluorous recycle reaction was considerably more efficient than the single pass DMC/TPFPP experiment, giving twice as much product with ×10 fewer moles of photocatalyst.

TABLE 1
Comparison between the F8/HFE-7500 recycle and the
TPFPP/DMC single pass experiment for oxidation of
citronellol over a period of 20 h.

| | F8/HFE- | |
|---|---|---|
| | 7500 | TPFPP/DMC^{c} |
| Amount of Citronellol | 1.32 [240] | 0.33 [60] |
| (mol [mL]) | | |
| Conversion (%) | 50^{a} | 100 |
| Yield of Products (4) + | 0.66 | 0.33 |
| (5) (mol) | | |
| Citronellol:PC^{d} | 5 000:1 | 1 000:1 |
| (mol:mol) | | |
| Apparent TON^{e} | 27 000 | 1 000 |
| Amount of PC at Start | 2.4 × 10⁻⁵ | 2.5 × 10⁻⁴ |
| (mol) | | |
| Amount of PC in HFE- | 6.5 × 10⁻⁶ | — |
| 7500 at End (mol) | | |
| Total volume of HFE- | 12 (+100)^{b} | — |
| 7500 (mL) | | |
| Recovery of F8 per | 88% | — |
| Recycle | | |
| Recovery of HFE-7500 | 55% | — |
| per Recycle | | |

^{a}The conversion of (3) to its peroxides, (4) + (5), was forced to ca. 50%.
^{b}The amount of HFE-7500 added during the reaction to replace that lost with CO₂. (i.e. 20 × 5 mL)
^{c}Dimethylcarbonate;
^{d}PC, photocatalyst;
^{e}TON, turnover number.

As in the initial flow experiments, nearly half the HFE-7500 was vented with the CO₂. The HFE-7500 could have been recovered from the CO₂ by using a more sophisticated separation device; however, for the purpose of the experiment, it was simpler to add more HFE-7500 at periodic intervals to maintain a reservoir volume of ca. 12 mL.

A twentyfold reduction in the amount of photocatalyst needed to make a given amount of product represents a significant reduction in the material cost of such a process.

It will be appreciated by those skilled in the art that the foregoing is a description of a preferred embodiment of the present invention and that variations in design and construction may be made to the preferred embodiment without departing from the scope of the invention as defined by the appended claims.

1. A method for photo-oxidising an organic substrate to form an organic product comprising the steps of:
a) mixing oxygen, a supercritical fluid, a photocatalyst, a liquid fluorous solvent and an organic substrate to form a mixture; and
b) irradiating the mixture to form an organic product.

2. A method for separating a photocatalyst from an organic product comprising the steps of:
a) providing a mixture comprising a supercritical fluid; an organic product; a fluorous solvent; a photocatalyst; and optionally an organic substrate and optionally oxygen; wherein the organic product, fluorous solvent, photocatalyst and optional organic substrate and optional oxygen are dissolved in the supercritical fluid; and
b) reducing the pressure of the mixture to a pressure below the critical pressure of the supercritical fluid in order to form a gaseous phase.

3. The method according to claim 1 comprising the step of reducing the pressure of the mixture to a pressure below the critical pressure of the supercritical fluid in order to form a gaseous phase.

4. The method according to claim 3 comprising the step of separating the gaseous phase from the fluorous solvent.

5. The method according to claim 3 comprising the step of separating the fluorous solvent from the organic product.

6. The method according to claim 5 wherein the fluorous solvent separated from the organic product comprises photocatalyst from the mixture.

7. The method according to claim 1 wherein the organic product and fluorous solvent are substantially immiscible.

8. The method according to claim 1 wherein the photocatalyst is soluble in the fluorous solvent.

9. The method according to claim 1 wherein the photocatalyst is soluble in the fluorous solvent rather than the organic product.

10. The method according to claim 1 wherein the photocatalyst, fluorous solvent, organic substrate and/or organic product are soluble in the supercritical fluid.

11. The method according to claim 1 wherein the fluorous solvent and the photocatalyst are recycled.

12. The method according to claim 1 wherein the mixture is single phase.

13.-14. (canceled)

15. The method according to claim 1 wherein the photocatalyst is fluorinated.

16.-18. (canceled)

19. The method according to claim 1 wherein the supercritical fluid is supercritical carbon dioxide.

20. An organic product manufactured using the method of claim 1.

21. (canceled)

22. A mixture comprising an organic substrate and/or organic product; oxygen; a supercritical fluid; a photocatalyst and a fluorous solvent.

23. The mixture according to claim 22 wherein the mixture is single phase.

24. The mixture according to claim 22 wherein the supercritical fluid is supercritical carbon dioxide.

25.-33. (canceled)
